(19) United States
(12) Patent Application Publication
(10) Pub. No.: US 2006/0172929 A1
(43) Pub. Date: Aug. 3, 2006

(54) Use of natriuretic peptides for the treatment of stature disorders related to the shox gene

(75) Inventors: Gudrun Rappold-Hoerbrand, Heidelberg (DE); Beate Haecker, Dossenheim (DE)

Correspondence Address: ROTHWELL, FIGG, ERNST & MANBECK, P.C., 1425 K STREET, N.W., SUITE 800, WASHINGTON, DC, 20005, US

(73) Assignee: Gudrun RAPPOLD-HOERBRAND, HEIDELBERG (DE)

(21) Appl. No.: 10/541,388

(22) PCT Filed: Jan. 12, 2004
(86) PCT No.: PCT/EP04/00134

§ 371 Date: Jul. 1, 2005

(30) Foreign Application Priority Data
Jan. 13, 2003 (EP) 03 000 728.0

(51) Int. Cl. A61K 38/17

(52) U.S. Cl. 514/12

(57) ABSTRACT

The invention relates to the use of natriuretic peptides (ANP or BNP) for the preparation of pharmaceutical compositions for the treatment of short stature in a subject being suspected of having a genetic defect in the human SHOX gene. Further, the invention relates to use of natriuretic peptides in combination with a growth protein and/or a SHOX protein for the preparation of pharmaceutical compositions for the treatment of patients having a SHOX gene disorder. The invention also relates to the use of natriuretic peptides (ANP and BNP) in combination with a SHOX protein for the preparation of pharmaceutical compositions for the treatment of patients with cardiovascular diseases. The invention also comprises an article of manufacture comprising packaging material and a pharmaceutical composition comprising natriuretic peptides.

This invention relates to the use of natriuretic peptides (atrial natriuretic peptide=ANP; brain natriuretic peptide=BNP) for the preparation of pharmaceutical compositions for the treatment of patients with short stature disorders related to the Short Stature Homeobox-containing gene (SHOX gene). More particularly, the invention relates to the use of ANP and/or BNP in combination with a growth protein and/or a SHOX protein for the treatment of a SHOX gene disorder, especially for the increasing or stimulating of human growth.

The Short Stature Homeobox-containing SHOX genes (SHOX I gene; SHOX II which is also referred to as SHOT gene) are described in WO 98/14568. The SHOX I gene is located in the pseudoautosomal region (PAR1) on the short arm of the X chromosome (Xp22.3) and Y chromosome (Yp11.3). Deletion or mutation of the SHOX I gene has been found in a number of patients with short stature, either idiopathic, or associated with Leri-Weill syndrome. Deficiency of the product of the SHOX I gene is believed to be the underlying cause of growth impairment in patients with Turner syndrome.

Turner syndrome is one of the most common genetic disorders with a prevalence of approximately 1 in 2500 liveborn females. One of the cardinal features is extreme short stature of more than 20 cm below the mean height of healthy adult women. Mean adult height of women with Turner syndrome ranges between 136.7 cm (Japan) and 146.9 cm (Germany). Most subjects suffer from gonadal dysgenesis with only a small percentage passing through puberty normally. In addition, many subjects show characteristic dysmorphic features with variable phenotypic penetrance, such as broad chest with widely spaced nipples, low posterior hairline, webbed neck, lymphedema, hyperconvex nails, and multiple cutaneous nevi. Renal and cardiac defects are also common. A number of skeletal abnormalities found in patients with Turner syndrome may be associated with reduced SHOX I expression during embryogenesis such as abnormal lower-to-upper leg/arm ratio (90%), micrognathia (60%), cubitus valgus (45%), high-arched palate (35%), short metacarpals (35%), genu valgum (30%), scoliosis (12%), and Madelung deformity (7%).

Natriuretic peptides (ANP and BNP) known from various origins, such as human, avian bovine or porcine origin, are peptides which are known to be regulators of natriuresis and vasodilation.

The growth hormones from man and from the common domestic animals are proteins of approximately 191 amino acids, synthesized and secreted from the anterior lope of the pituitary gland. Human growth hormone consists of 191 amino acids. Growth hormone is a key hormone involved in the regulation of not only somatic growth, but also in the regulation of metabolism of proteins, carbohydrates and lipids. The major effect of growth hormone is to promote growth. The organ systems affected by growth hormone include the skeleton, connective tissue, muscles, and viscera such as liver, intestine, and kidneys.

Studies conducted by a number of manufacturers of somatropin (recombinant human growth hormone, rhGH) have demonstrated that human growth hormone is effective in increasing the final height of subjects with Turner syndrome. Turner syndrome has been registered as an approved indication of somatropin therapy worldwide in most countries, including the United States, based on data that show an increase in growth velocity and an improvement of final height. The cause of short stature in Turner syndrome and in other subjects with SHOX I defect with or without skeletal dysplasias (SHOX I disorder) is haploinsuffliciency of the SHOX I gene.

Surprisingly, it has now been found that the natriuretic proteins ANP and BNP can be used for the preparation of pharmaceutical compostions for the treatment of subjects being suspected of or actually having a genetic defect in the SHOX gene. The treatment comprises administering to such a subject a pharmaceutically active amount of a natriuretic peptide (ANP and/or 1BNP). In a preferred embodiment of this method, the subject is a human subject. Further, as a preferred embodiment of the present invention, natriuretic peptides, especially BNP, and preferably human BNP, can be used in combination with a growth hormone, especially with human growth hormone (hGH) and/or in combination with a SHOX protein.

The invention also provides an article of manufacture comprising packaging material and a pharmaceutical composition comprising at least one of the natriuretic peptides ANP and/or BNP contained within the packaging material. This pharmaceutical composition is therapeutically effective for treatment of short stature due to a SHOX gene disorder, and the packaging material comprises a label which indicates that the natriuretic peptides can be administered to a subject with a SHOX gene disorder. In a preferred embodiment of this article, the article of manufacture comprises additionally a pharmaceutical composition comprising a growth hormone, especially human growth hormone. The growth hormone can be either included in the same pharmaceutical composition as the natriuretic peptide(s) or, alternatively, can also be formulated in a separate pharmaceutical composition. The packaging material comprises a label which indicates that the natriuretic peptide(s) is/are effective in increasing growth velocity of subjects with a SHOX I gene disorder.

### BRIEF DESCRIPTION OF THE DRAWINGS

FIG. 1: BNP expression after induction of SHOX

FIG. 1A: Semiquantitative RT-PCR with SHOX or BNP specific primers was performed on total RNA isolated from U2OS-SHOX or U2OS-STM cells 48 hours after induction (ind) of protein expression and on RNA from uninduced control cells (unind). BNP is detectable only upon induction of the full length SHOX protein in the induced U2OS-SHOX cells.

FIG. 1B: (not shown) Total RNA was extracted from the inducible cell line U2OS-SHOX which expressed SHOX at 0, 12, 24, 36, 48 and 72 hours or from uninduced U2OS control cells. Concentration of BNP mRNA was determined by quantitative RT-PCR carried out in duplicate using GAPDH as a standard. BNP mRNA levels (diamonds) increased significantly with time compared to the uninduced cells (squares).

FIG. 2: Electromobility Shift Assay (EMSA) of the proximal SHOX binding site BNP-600.

FIG. 2A: 10 fmol of ³²P-radiolabelled double-stranded oligonucleotide containing the putative proximal binding site of SHOX was incubated with 0, 0.05, 0.5 and 3 μl purified SHOX-GST (250 nM). Monomeric binding of SHOX-GST could be observed with volumes of 0.05 and 0.5 μl, an increase in SHOX-GST concentration led to the formation of homeodimers.

Competition: Incubation of 1 μl of SHOX-GST with 10 fmol radiolabelled oligonucleotide and an increasing (0, 50×, 150×, 500× and 1000×) excess of unlabelled oligonucleotide resulted in a decrease in signal intensity.

Supershift: anti-SHOX antibody (AB) was added to the oligonucleotide-SHOX-GST complex. An additional shift of the monomeric SHOX-GST-Oligonucleotide complex could be observed, which had not been seen in the controls, indicating the binding of the AB.

M: monomeric binding; D: dimeric binding; SS: supershift. GST: purified GST-tag alone; -: no protein extract added.

FIG. 2B: Sequence specificity of the binding.

To test the sequence specificity of the SHOX DNA binding SHOX-GST was incubated with oligonucleotides containing artificially introduced mutations in the putative SHOX binding site. Nucleotides differing from the wild type sequence (Wt) are highlighted in green (BNP-600a, BNP-600b). As the number of mutated nucleotides increased, binding was strongly reduced (BNP-600a) or completely disappeared (BNP-600b).

FIG. 3: Electromobility Shift Assay (EMSA) of the distal SHOX binding site BNP-1220.

FIG. 3A: 10 fmol of ³²P-radiolabelled double stranded oligonucleotide containing the putative distal binding site of SHOX was incubated with 0, 0.05, 0.5 and 3 μl purified SHOX-GST (250 nM). Monomeric binding of SHOX-GST could be observed with volumes of 0.05 and 0.5 μl, an increase in SHOX-GST concentration led to the formation of homeodimers.

Competition: Incubation of 1 μl of STM with 10 fmol radiolabelled oligonucleotide and an increasing (0, 50×, 150× and 1000×) excess of unlabelled oligonucleotide resulted in a decrease in signal intensity.

Supershift: anti-SHOX antibody (AB) was added to the oligonucleotide-SHOX-GST complex. An additional shift of the monomeric SHOX-GST-Oligonucleotide complex could be observed, which was not seen in the controls, indicating the binding of the AB.

M: monomeric binding; D: dimeric binding; SS: supershift. GST: purified GST-tag alone; -: no protein extract added.

FIG. 3B: Sequence specificity of the binding.

To test the sequence specificity of the SHOX DNA binding, SHOX-GST was incubated with oligonucleotides containing artificially introduced mutations in the putative SHOX binding site. Nucleotides differing from the wild-type sequence (Wt) are highlighted in green (BNP-1220a, BNP-1220b, BNP-1220c). As the number of mutated nucleotides increased, binding was strongly reduced (BNP-1220a, BNP-1220b) or completely disappeared (BNP-1220c).

FIG. 4:

FIG. 4A: Genomic locus of BNP. Exons are represented by blue boxes, start and stop codon are indicated. Sequences of the putative SHOX binding sites (BNP-1220 and BNP-600) are shown.

FIG. 4B: Reporter constructs for the activity analysis of the BNP regulatory region. Putative SHOX binding sites in the regulatory region of BNP are indicated. The regulatory region was inserted in forward (BNP for) and reverse (BNP rev) orientation. The construct p3XG was generated by insertion of an experimentally determined SHOX binding site in front of the SV40 core promoter (Rao et al., 2000).

FIG. 4C: Luciferase activity after induction of SHOX. Reporter constructs were transiently transfected into U2Os-SHOX or U2OS-STM cells and luciferase activity was determined after 48 hours of SHOX or SHOX-STM induction. With BNP for a 10-fold increase was observed upon induction compared to uninduced control cells. BNP rev revealed an 8-fold and p3XG a 2-fold increase of luciferase activity. No significant changes in luciferase activity were obtained for the negative control vector pGL3promoter. All experiments were performed in triplicate. The bars represent the mean values of two independent experiments.

### DETAILED DESCRIPTION

Patients with haploinsufficiency of the SHOX I gene present short stature. This patient group consists of Turner, Leri-Weill and Langer syndrome patients and patients with idiopathic short stature (Rappold and Blaschke, 2000). It was found that 2.4% of patients with idiopathic short stature present a SHOX I gene defect (Rappold et al, 2002) which would imply a population prevalence of at least 1 in 2000 children. The prevalence of Turner syndrome is 1 in 2500 girls or 1 in 5000 children (Rosenfeld et al, 1996). Females with Turner syndrome are frequently treated with growth hormone, despite the absence of growth hormone deficiency. Clinical studies have demonstrated that recombinant human growth hormone is effective and generally safe in increasing the final height in children with Turner syndrome (Carel et al, 1998; Rosenfeld et al, 1998; Sas et al, 1999). As growth hormone is effective in the treatment of idiopathic short stature and Turner syndrome, it has been hypothesized that it may also improve the growth rate and final height in children with a SHOX gene defect without Turner syndrome (Rao et al, 1997).

Despite this progress, the development of more specific and effective concepts for growth therapy on growth failures based on the SHOX I gene is needed (Rao et al, 1997). One route in the search for a specific therapy based on the primary SHOX genetic defect is the identification of downstream targets. By microarray analysis and quantitative RT-PCR of in vitro and in vivo cell culture systems using a tetracycline inducable SHOX wildtype and a C-terminally mutated construct, the present inventors were able to show that overexpression of SHOX leads to an impressive upregulation of BNP. It could also be demonstrated, using DNA binding and EMSA assays with wildtype and artificially mutated SHOX DNA binding sites, that the SHOX I protein binds to two cis-acting elements in the 5′flanking region of BNP and induces the expression of BNP in cultured cells. As the homeo-domain of SHOX I and SHOX II (SHOT) are identical, the binding of the homeo-domain may affect identical target genes (ANP, BNP). BNP, while mainly secreted by cardiac ventricle myocytes, has been recently shown to be also secreted in high amounts in bone marrow stromal cells where the maximum expression of SHOX I has been previously described (Bordenave et al, 2002).

Natriuretic peptides represent key regulators of natriuresis and vasodilation. Up to now, four ligands (ANP, BNP, CNP and DNP and three receptors (GC-A, GC-B and Clearance receptor) have been described (Dhingra et al, 2002). The natriuretic peptides are structurally similar, but genetically distinct peptides that have diverse actions on cardiovascular, renal and endocrine homeostasis. Atrial natriuretic peptide (ANP) and brain natriuretic peptide (BNP) are cardiac (endocrine) hormones of myocardial cell origin predominantly produced by the atrium and ventricle, respectively and regulate blood pressure and body fluid volume. CNP (cardiac natriuretic peptide) is principally a paracrine factor in the brain and periphery. ANP and BNP bind to the natriuretic peptide receptor (NPR-A) which, via 3′ 5′-cyclic guanosine monophosphate (cGMP), mediates natriuresis, vasodialation and renin inhibition. CNP lacks natriuretic action but also possesses vasodilating and growth inhibiting effects via the guanyl cyclase linked natriuretic peptide-B (NPR-B) receptor. All three peptides are cleared by natriuretic peptide-C receptor (NPR-C) and degraded by neutral endopeptidase. Recently, a fourth member of the natriuretic peptide, dendroaspsis natriuretic peptide (DNP) has been reported to be present in human plasma and atrial myocardium.

Over the last decade, brain natriuretic peptide (BNP) emerged as a cardiac hormone of considerable clinical interest in diagnosis, prognosis and treatment of patients with heart failure, hypertension and other cardiac disease (Burger and Burger, 2001; Bettencourt, 2002). The diagnostic potential of BNP is now well established both in patients with suspected heart failure as well as in patients with asymptomatic left ventricular systolic dysfunction. The prognostic information obtained from BNP levels in heart failure and acute myocardial infarction patients seems even more promising. Nesiritide (Natrecor® manufactured by Scios, Inc) is a synthetic peptide, homologous to endogenous BNP. It is a balanced vasodilator with diuretic and natriuretic properties. It decreases the elevated levels of neurohormones resulting from activation of the sympathetic and renin-aldosterone systems in heart failure. In clinical trials involving more than 2000 patients with heart failure nesiritide has been shown to produce a potent, dose-related vasodilator effect. The safety profile has been excellent with a dose-dependent hypotension as the major side effect (Bettencourt, 2002).

Surprisingly, an increase of SHOX expression leads to an upregulation of ANP and BNP expression. Haploinsufficiency in SHOX deficient patients consequently should lead to a downregulation of BNP expression. Mice overexpressing BNP do not only present a skeletal overgrowth and kyphosis but also show a reduction in blood pressure (hypotonia) and present defects in the cardiovascular and renal physiology (Suda et al, 1998). Interestingly, females with Turner syndrome present for some of the observed clinical features the ‘opposite phenotype’ such as short stature and scoliosis, an increase in blood pressure (hypertonia) and further heart and renal abnormalities. The present results indicate that natriueretic peptides represent targets of SHOX proteins. Brain natriuretic peptides (BNP) and derivatives thereof are thus useful for the treatment of SHOX deficient children. Natriuretic peptides (ANP, BNP) are able to compensate growth failure in a more direct and specific way than growth hormone. Especially, brain natriuretic peptides (BNP) is useful to improve the growth rate and/or the final height in subjects with a SHOX defect, especially to increase the final height in children with or without Turner syndrome.

As used herein, a “subject having a SHOX gene disorder” (or a “subject being suspected of having a SHOX gene disorder”) is defined as a subject, either actually having a mutation (or a subject to be investigated by means of appropriate diagnostic methods of the possibility of having a SHOX gene disorder), with a mutation which reduces expression or activity of a product (e.g., mRNA or polypeptide or an activity of a polypeptide, such as a binding activity) encoded by the Short Stature Homeobox-containing (SHOX I) gene on at least one chromosome of the subject, which gene in the human genome is located in the pseudoautosomal region (PAR1) on the short arm of the X chromosome (Xp22.3) and Y chromosome (Yp11.3). Methods for screening for SHOX gene defects are described, for instance, in published PCT application WO 98/14568. The mutation of the SHOX genes may comprise a deletion or other mutation of all or any part of the SHOX genes, as identified by DNA analysis or other appropriate molecular technique, or a mutation elsewhere in the genome of the subject which nevertheless reduces expression and/or activity of a SHOX gene products. Subjects with SHOX disorder include those with and without Leri-Weill syndrome.

Subjects with Turner syndrome are defined as follows: Females whose karyotype contains a documented abnormality of the X chromosome involving the short arm (for example, 45,X; 46,X,Xp-; 46X,i[Xq]). Female subjects with a partial deletion of the short arm of the X-chromosome are not defined as having Turner syndrome, if the deletion is located distal to the gene for ocular albinism (OA1) at the junction between Xp22.2 and Xp22.3. Instead, they are defined as having a SHOX I disorder.

In addition, a subject having a “SHOX gene disorder” as defined herein also has an abnormally short stature, according to standard measures known in the art, such as may be observed in subjects with growth hormone deficiency. However, subjects having a SHOX gene disorder are not growth hormone deficient by standards known in the art. For instance, a subject with a SHOX gene disorder has a peak growth hormone level greater than 7 ng/ml or 14 mU/L. For treatment, a subject with a SHOX gene disorder is considered to have abnormally short stature if the subject has a chronological age of at least 3 years, bone age of less than 10 years for boys and less than 8 years for girls, and height below the 3rd percentile or height below the 10th percentile and growth velocity below the 25th percentile, for an appropriate age-and-sex-matched ‘normal’ reference population based upon local standards. For this exemplary study, subjects with a SHOX disorder also are prepubertal (for girls, Tanner stage 1 with respect to breast development; for boys, Tanner stage 1 with respect to genital development and testicular volume of no more than 2 ml).

In the context of the present invention, the term “natriuretic peptide(s)” (ANP, BNP) is used to represent any natriuretic peptide known from prior art or from any origin, such as avian, bovine, human or porcine natriuretic peptide, human natriuretic peptides being most preferred. The natriuretic peptides used in accordance with the present invention may be native natriuretic peptides isolated from a natural source, or a natriuretic peptide produced by recombinant techniques. The natriuretic peptide(s) may also be a truncated form of the native natriuretic peptide(s) wherein one or more amino acid residues has (have) been deleted; an analogue thereof wherein one or more amino acid residues in the native molecule has (have) been substituted by another amino acid residue, preferably a natural amino acid residue, as long as the substitution does not have any adverse effect such as antigenicity or reduced action; or a derivative thereof, e.g having an N- or C-terminal extension such as Met-ANP or Met-BNP. The preferred natriuretic peptide according to this invention is human brain natriuretic peptide.

The term “dose” of ANP or BNP refers to that amount that provides therapeutic effect in an administration regimen. The natriuretic peptides are formulated for administering a dose effective for increasing growth rate or final height of a subject having a SHOX I or SHOX II gene disorder, for instance, a dose similar and known to one effective dose also in the treatment of cardiovascular diseases.

In the context of the present invention, the term “growth hormone” may be growth hormone from any origin such as avian, bovine, equine, human, bovine, porcine, salmon, trout or tuna growth hormone, preferably bovine, human or porcine growth hormone, human growth hormone being most preferred. The growth hormone used in accordance with the invention may be native growth hormone isolated from a natural source, e.g. by extracting pituitary glands in a conventional manner, or a growth hormone produced by recombinant techniques, e.g. as described in E. B. Jensen and S. Carlsen in Biotech and Bioeng. 36, 1-11 (1990). The “growth hormone” may also be a truncated form of growth hormone wherein one or more amino acid residues has (have) been deleted; an analogue thereof wherein one or more amino acid residues in the native molecule has (have) been substituted by another amino acid residue, preferably a natural ammo acid residue, as long as the substitution does not have any adverse effect such as antigenicity or reduced action; or a derivative thereof, e.g having an N- or C-terminal extension such as Met-hGH. The preferred growth hormone is human growth hormone (hGH).

In general, natriuretic peptides can be administered by any feasible administration route, such as formulations for parenteral administration. Such parenteral formulations are prepared containing amounts of natriuretic peptides known from prior art, for example, in the range of about 0.1 mg/ml-40 mg/ml, preferably from about 1 mg/ml to about 25 mg/ml, or to about 5 mg/ml, calculated on the ready-to-use formulation. For use of these compositions in administration to human beings suffering from SHOX disorder, for example, these formulations contain from about 0.1 mg/ml to about 10 mg/ml, corresponding to the currently contemplated dosage regimen for the intended treatment. The concentration range may be varied by the physician supervising the administration

A natriuretic peptide can typically be administered parenterally, preferably by subcutaneous injection, by methods and in formulations well known in the art.

Natriuretic peptides can be formulated with typical buffers and excipients employed in the art to stabilize and solubilize proteins for parenteral administration.

With respect to the growth protein, the similar administration route or formulations as mentioned before for natriuretic peptide can be used. Appropriate formulations are described, for example, in U.S. Pat. No. 5,612,315, disclosing pharmaceutical growth hormone formulations, and U.S. Pat. No. 5,851,992, disclosing human growth hormone formulations which may be used to treat a patient with a disorder associated with growth hormone deficiency.

Art recognized pharmaceutical carriers and their formulations are described in Martin, “Remington's Pharmaceutical Sciences,” 15th Ed.; Mack Publishing Co., Easton (1975). A BNP, growth hormone or SHOX protein can also be delivered via the lungs, mouth, nose, by suppository, or by oral formulations, using methods known in the art.

The natriuretic peptide(s), the growth protein or SHOX protein (either SHOX A and/or SHOX B, as described in WO 98/14568) can be administered regularly (e.g., once or more each day or week), intermittently (e.g., irregularly during a day or week), or cyclically (e.g., regularly for a period of days or weeks followed by a period without administration). Preferably the aforementioned proteins are administered once daily for at least about one year, more preferably at least about three years, and most preferably for at least about six or seven years.

The present invention also encompasses articles of manufacture comprising packaging material and a pharmaceutical composition comprising a natriuretic peptide contained within the packaging material. This pharmaceutical composition is therapeutically effective for treatment of short stature due to a SHOX I gene disorder, and the packaging material comprises a label which indicates that the natriuretic peptide, possibly in combination with a growth protein or a SHOX protein, can be administered to a subject with a SHOX gene disorder.

For instance, an article of manufacture of this invention may comprise a kit including pharmaceutical compositions to be used in the methods of the present invention. The kit can contain a container, such as a vial or cartridge for an injection pen, which contains a formulation of a natriuretic peptide, growth hormone or SHOX protein and suitable carriers, either dried or in liquid form. The kit further includes instructions in the form of a label on the vial or cartridge and/or in the form of an insert included in a box in which the vial or cartridge is packaged, for the use and administration of a natriuretic peptide, the growth hormone or SHOX protein composition. The instructions can also be printed on the box in which the vial or cartridge is packaged. The instructions contain information such as sufficient dosage and administration information so as to allow a worker in the field or a human subject to administer the drug, as is customary in most locales. The article of manufacture is produced and assembled as a fixed pharmaceutical package unit by pharmaceutical companies for sale in, for example, hospitals or drug stores, or for sale to physicians.

The example which follows are illustrative of the invention and are not intended to be limiting.

### EXAMPLES

### Example 1

Overexpression of SHOX Leads to an Upregulation of BNP

To identify putative SHOX target genes, an inducible cell culture model was used consisting of two stably transfected osteosarcoma U2Os cell lines, U2Os-SHOX and U2Os-STM (Rao et al, 2001). Upon tetracycline induction U2Os-SHOX expresses the full-length wildtype SHOX protein while U2Os-STM expresses a C-terminally truncated mutant, SHOX-STM (Rao et al., 2001). RNA from both cell lines was harvested after 12 and after 24 hours of SHOX induction and used to hybridize the Affymetrix Human cDNA (Hu95A) microarrays. These arrays allowed the interrogation of 12 000 RNAs from 8900 different human genes.

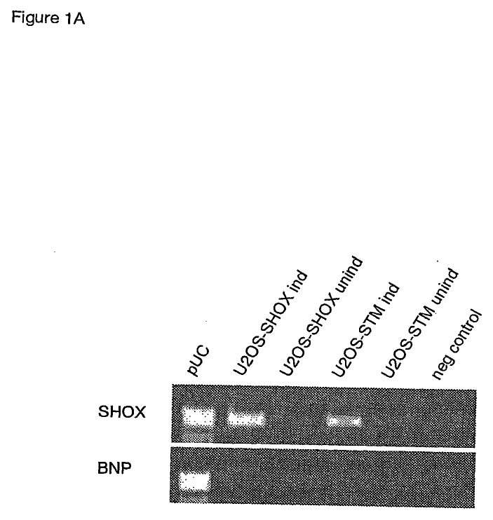

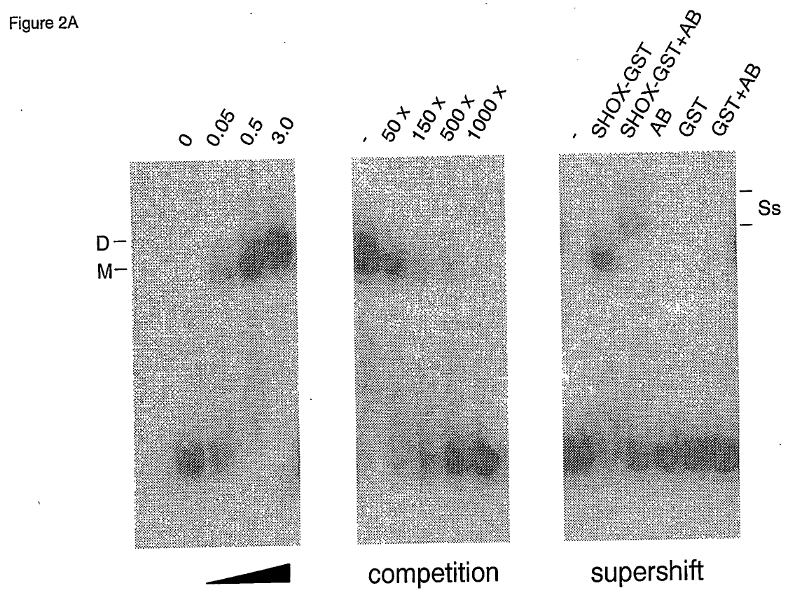

The maximum induction of expression above a threshold of 10 after 12 hours of SHOX induction was only detected for a single gene. An upregulation of 13.6 fold was seen in the mRNA level of the brain natriuretic protein (BNP) gene (Seilhamer et al, 1989; accession number M31776) which further increased to 17.1 fold after 24 hours of SHOX expression. This increase in BNP mRNA was not detectable in the mutant which presented a ˜2.5 fold change after 12 hours and a 1.1 fold change after 24 hours. The differential expression of BNP was confirmed by semiquantitative and quantitative RT-PCR using RNA from a different preparation. The RT-PCR result corresponded very well with the microarray data, demonstrating that BNP levels were increased after induction of SHOX protein expression, but not upon expression of the SHOX-STM which lacks transactivating properties (FIG. 1A). In a time course experiment, RNA after 0, 12, 24, 36, 48 and 72 hours of SHOX induction was analyzed for BNP expression by quantitative RT-PCR. These experiments confirmed a significant increase of BNP expression after only 24 hours (FIG. 1B). These results strongly suggest that the upregulation in BNP mRNA is specifically due to the transactivating properties of SHOX. Furthermore, the relatively rapid response of BNP after SHOX induction points to the possibility of BNP being a direct target of SHOX.

### Example 2

The SHOX Protein Binds to cis Acting Elements in the Regulatory Region of BNP

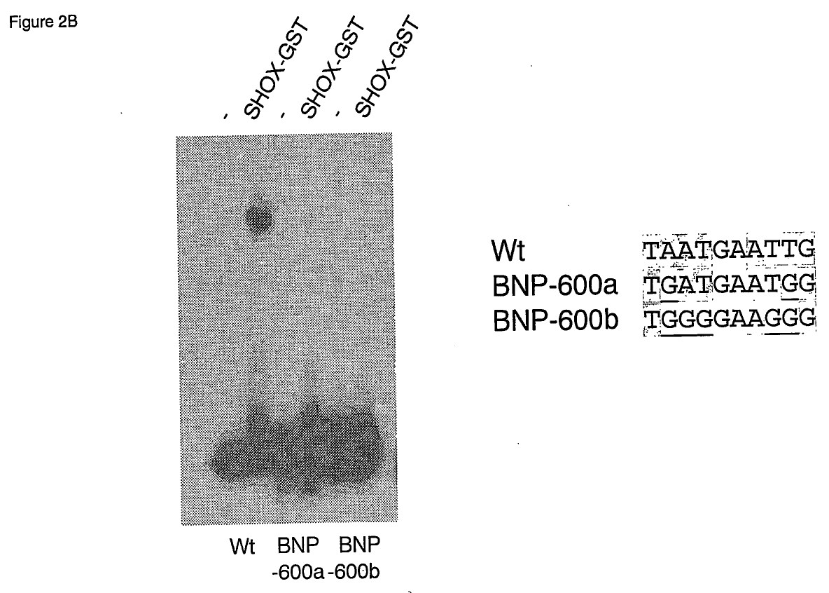

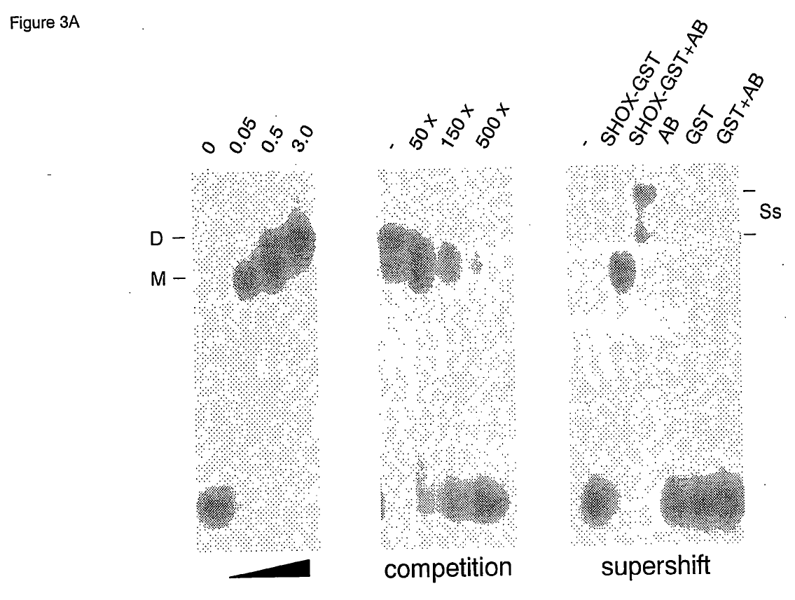

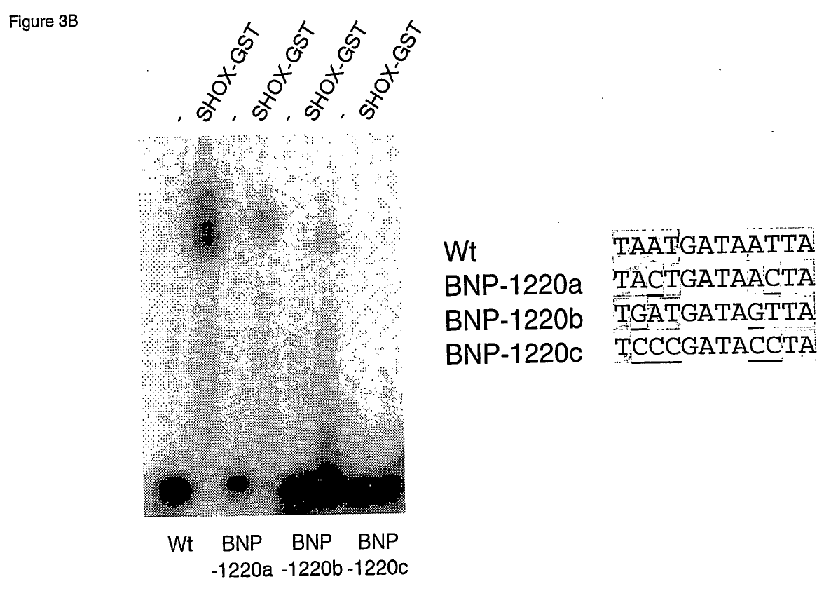

Paired-related homeodomain proteins including SHOX preferentially bind to a palindromic sequence TAAT(N)ₙATTA, where two palindromic TAAT sequences are separated by two to four less significant nucleotides (N) (Wilson et al., 1993). This has also experimentally been demonstrated to hold true for SHOX (Rao et al, 2001). Interestingly, two of those palindromic sequences are present in the 5′flanking regulatory region of the BNP gene (accession number D.16641). The proximal one, TAATGAATTG, is 600 nucleotides upstream of the mRNA, further referred to as BNP600, and the distal one, TAATGATAATTA, is −1220 nucleotides upstream, further referred to as BNP-1220. To demonstrate specific in vitro interaction of SHOX to these DNA sequences, we have performed electromobility shift assays (EMSA) with BNP-600 and BNP-1220 specific oligonucleotides. Electromobility shifts were observed at low protein concentration of 0.5 μM for both predicted binding sites, with a slightly higher affinity towards BNP-1220 as compared to BNP-600. Rise of the SHOX concentration to 0.5 and 3 μM led to the formation of homodimeric complexes. Again, a preference of the SHOX protein for BNP-1220 was observed which resulted in a dimeric complex at lower concentrations compared to BNP-600. In competition experiments no difference between the two sequences BNP-600 and BNP-1220 was detected; for both DNAs a minimum of 500-fold excess of cold oligonucleotide was necessary to completely inhibit the binding of SHOX. In supershift experiments a supershift of the signal was generated only in the presence of both SHOX and rabbit anti-human SHOX-3 antibody (AB) (FIGS. 2A and 3A). Furthermore, substitution of two nucleotides within the palindromic sequence almost completely abolished SHOX binding. Replacement of five nucleotides in the palindromic sequence led to an entire loss of SHOX binding, indicating the sequence specificity of this protein-DNA interaction. These data strongly support the existence of (at least) two binding sites recognized by the SHOX protein in the regulatory region of BNP. BNP therefore is a direct target for the transcription factor SHOX.

### Example 3

The SHOX Protein Induces the Expression of BNP in Cultured Cells

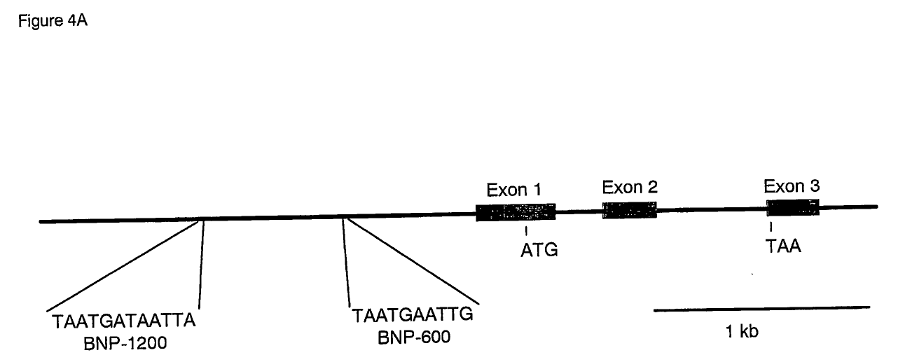

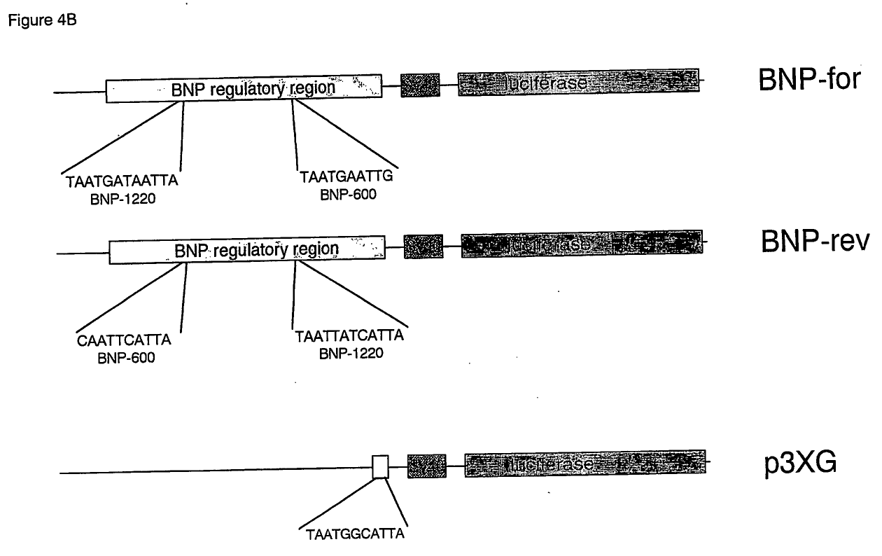

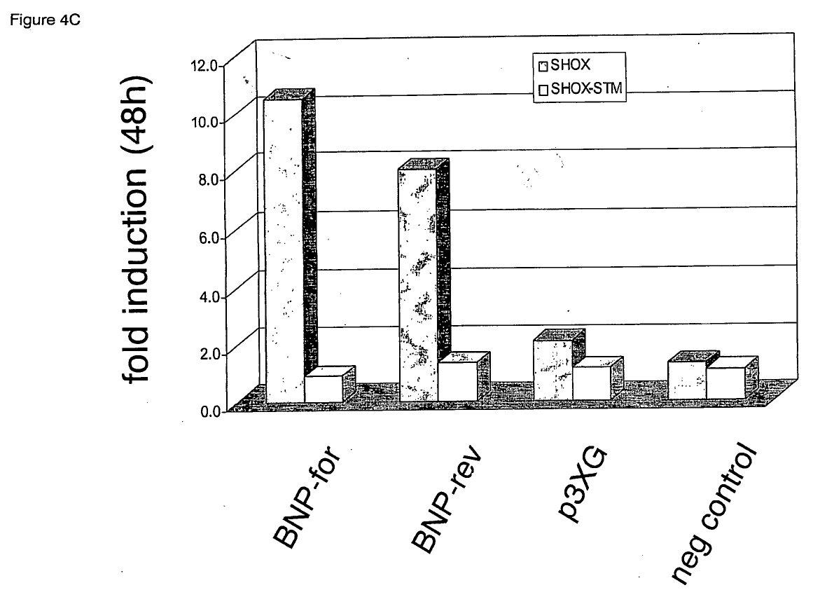

To verify the binding of the SHOX protein to its putative responsive elements BNP-600 and BNP-1200 consequently activating BNP expression in vivo, reporter plasmids were generated containing the 5′ flanking region of the BNP gene in forward and reverse orientation. As shown in FIG. 4, transient transfection of U2Os-SHOX cells with the BNP—for construct revealed a 10-fold increase in luciferase activity after expression of the SHOX protein for 48 hours. The increase in the reporter protein was lower compared to the BNP mRNA levels after SHOX induction as previously determined in time course experiments. This is probably partly due to the leakage of the TET-SV40 core promoter system resulting in relatively high background levels of reporter protein expression. An 8-fold increase of luciferase activity was also observed in BNP-rev, indicating that the SHOX responsive elements do not show orientation specificity. The approximately 4-to 5-fold higher level of induction with the BNP- for and -rev compared to the p3XG (a previously experimentally determined SHOX binding site) suggests a potentiation of the transactivating effect by the interaction of SHOX with other factors. No increase in luciferase activity was obtained when the same constructs were transfected into U2Os-STM confirming that induction of the reporter protein expression depends on SHOX transactivating activity. The specific BNP upregulation upon the induction of the full-length SHOX but not of the C-terminally truncated SHOX demonstrates SHOX as a transcriptional activator of BNP expression in U2Os cells.

### Example 4

In vivo Results Using Chromatin Fixation and Growth Plate Sections

a) Using chromatin fixation (Weinmann et al., 2002), a binding of the SHOX protein to the regulatory region (promotor) of the BNP gene could be demonstrated in vivo. Hereby U2OS cells, which express the SHOX gene for 48 hours, were treated with formaldehyde to allow fixation of the DNA binding proteins to the DNA. The cell chromatin was isolated and the fixed DNA precipitated together with SHOX through addition of a SHOX specific antibody. For controls, premium serum or water was used instead of the antibody. The precipitated DNA was analyzed by quantitative RT-PCR for an accumulation of SHOX binding sites in the putative regulatory region of the BNP gene. For BNP-600, an accumulation factor of 428, for BNP-1220 an accumulation factor of 301 was obtained.
b) Immunohistochemical analysis on growth plate sections of a patient with idiopathic tall stature revealed BNP expression in hypertrophic chondrocytes. The SHOX protein has been shown previously to be expressed in the same cells in the chondrocytes in the growth plate.

### Example 5

### Materials and Supplies

Cells and Media

Generation of the human T-Rex U2OS osteosarcoma cell line (R712-07; Invitrogen) stably transfected with the gene for the wild type or a C-terminally truncated SHOX protein in a tetracycline inducible system (ST or STM respectively) has been described previously (Rao et al., 2001). Cells were cultured in DMEM containing glucose at 4.5 g/l under selection with hygromycin (10 μg/ml) and zeozin (100 μg/ml) at 37° C. with 5% CO₂. Induction of SHOX or SHOX-STM expression was obtained by addition of the tetracycline analogous doxycycline (4 μg/ml).

Escherichia coli was cultured in LB medium containing 5 g/l yeast extract, 10 g/l tryptone and 5 g/l NaCl at 37° C. with aeration. Strains harboring plasmids for expression of GST fusion proteins were kept under selection with 100 μg/ml Ampicillin. To induce expression of recombinant proteins isopropyl-β-D-thiogalactoside (IPTG) was added to a final concentration of 1 nM.

Expression and Purification of GST Fusion Proteins

E. coli harboring pGEX constructs containing the gene for a N-terminal GST-SHOX-STM fusion protein (SHOX aminoacid positions 1-194, further referred to as SHOX-GST) or the empty vector were grown at 28° C. to an optical density of 0.5 at 600 nm. Gene expression was then induced for 5 hours. Cells were harvested by centrifugation at 4500 g for 10 min, washed once with icecold NETN buffer (20 mM Tris/HCl pH 8.0, 100 mM NaCl, 1 mM EDTA, 0.5% NP-40) and resuspended in 1/50 volume NETN buffer. After 4 rounds of sonication with a sonicator™ cell disruptor (Heat Systems—Ultrasonics Inc.) for 5 min insoluble proteins were pelleted by spinning the disrupted bacteria for 30 min at 13 000 rpm at 4° C. in an Heraeus benchtop microcentrifuge. To allow binding of the GST fusion protein, the supernatant was incubated for 20 min at room temperature with 1/20 Glutathione Sepharose beads suspended in NETN buffer. After collecting the beads by centrifugation for 10 s at 6000 rpm in an Eppendorf benchtop microcentrifuge fusion proteins were eluted with one bead volume of 10 mM reduced glutathione in 50 mM Tris/HCl pH 8.0 and validated for integrity and purity by SDS PAGE analysis on a 10% polyacrylamide gel. Concentration of the protein solution was determined with the Pierce BCA* Protein Assay according to the manufacturers instructions and samples were stored in aliquots at −80° C.

Affymetrix Hu95A GeneChip Hybridisation

The Hu95A GeneChip (Santa Clara, Calif., USA) contains more than 12 000 probe sets corresponding to 8900 specific human genes (UniGene Build 139). Total RNA for the hybridization was isolated from human U2OS cells expressing either SHOX or STM. Cells were grown in 145 mm tissue culture dishes and gene expression was induced at 30% confluence for 12 or 24 hours. As a negative control uninduced cells were also harvested at corresponding time points. After RNA preparation using the Qiagen RNeasy® Midi Kit the RNA was quantified, and validated for integrity by gel electrophoresis. Induction of the SHOX/STM gene expression was verified by first strand cDNA synthesis using the Superscript™ First-Strand Synthesis System for RT-PCR from GibcoBRL® according to the manufacturer's protocol and semiquantitative RT-PCR. Target synthesis, hybridization of the samples to the Hu95A GeneChips, post hybridization staining, scanning of the stained chips and processing of the data files using the MAS 4.0 and MAS 5.0 software were performed at the RZPD in Berlin.

Real Time Polymerase Chain Reaction

For quantitative real time (RT)-PCR analysis RNA extracted from cells was reverse transcribed as described before. The resulting first strand cDNA was used as template in PCR reactions. Primers for PCR were selected using the Primer3 software and checked for specificity by NCBI BLAST of the human genome. In addition to melting curve analysis the resulting PCR products were analyzed for specificity on agarose gels. The following primer pairs were used in PCR experiments:

GAPDH:
ACCACAGTCCATGCCATCAC, TCCACCACCCTGTTGCTGTA;
SHOX:
ATGGAAGAGCTCACGGCTTTTGTATCC, GAAGAGTCGCTCGAGCTCGTT
C;
BNP:
TTCTTGCATCTGGCTTTCCT, ACCGTGGAAATTTTGTGCTC.

Quantitative PCRs were performed with the LightCycler—FastStart DNA Master SYBR green I Kit (Roche Diagnostics GMBH, Mannheim, Germany), PCR conditions as follows: Denaturation of DNA and activation of the polymerase at 94° C. for 10 min; 45 cycles of a touch down PCR with 15 s denaturation at 94° C., 10 s annealing at 65° C. with a decrease of 1° C. per cycle to a final annealing temperature of 60° C., elongation at 72° C. for 30 s. Expression of genes was analyzed on the LightCycler from Roche Molecular Systems with the LightCycler Software 3.5. For normalization we used the expression of the housekeeping gene GAPDH.

Electromobility Shift Assay (EMSA)

To create double stranded DNA for mobility shift assays two complementary oligonucleotides were annealed, generating 5′ overhangs on each side to permit radiolabelling using the Taq polymerase. The following oligonucleotides were used in the shift experiments (only the forward strand of the probes are given, putative binding site in bold, mutagenized nucleotides underlined):

BNP-1220 Wt:
TAATCACCAGGCCACCTGCTAATGATAATTAGATCATGGGTGGTCAGATG
BNP-1220a
TAATCACCAGGCCACCTGCTACTGATAACTAGATCATGGGTGGTCAGATG
BNP-1220b:
GGGTCACCAGGCCACCTGCTGATGATAGTTAGATCATGGGTGGTCAGATG
BNP-1220c:
GGGTCACCAGGCCACCTGCTCCCGATACCTAGATCATGGGTGGTCAGATG
BNP-600Wt:
TTCCTGGTCATACCCAGGCTTTTAATGAATTGCCACTGGGGAATCAGCAT
BNP-600a:
GGGTTCCTGGTCATACCCAGGCTTTTGATGAATGGCCACTGGGGAATCAG
CAT;
BNP-600b:
GGGTTCCTGGTCATACCCAGGCTTTTGGGGAAGGGCCACTGGGGAATCAG
CAT.

Gel Shift reactions contained 15 mM Hepes pH 7.5, 150 mM NaCl, 1 mM EDTA, 0.5 mM DTT, 0.05% NP-40, 7.5% glycerol, 0.25 mg/ml bovine serum albumin, 1.5 μg poly(dI-dC) and the appropriate ³²P-labelled probe. After addition of protein extract the samples were incubated for 10 min at room temperature. Separation was performed on a 5% nondenaturing polyacrylamide gel with 0.25× TBE at 12 V/cm at room temperature for 50 min. Gels were then dried and exposed overnight at −80° C. All gel shifts were performed with bacterially expressed and purified SHOX-GST which has previously been shown to have DNA-binding properties identical to the eukariotically expressed SHOX protein (Rao et al. 2001). In competition experiments, varying amounts of unlabelled competitor DNA were added to the samples preceeding addition of protein extracts. In supershift experiments, 3 μg of anti-human SHOX-3-specific antibody were applied to the samples and preincubated for 15 min on ice prior separation for 2 h at 12 V/cm and 8° C.

### REFERENCES

The invention has been described with reference to various specific and preferred embodiments and techniques. However, it should be understood that many variations and modifications may be made while remaining within the spirit and scope of the invention. All publications and patent applications in this specification are indicative of the level of ordinary skill in the art to which this invention pertains.

Bettencourt, P. (2002) Brain natriuretic peptide (nesiritide) in the treatment of heart failure. Cardiovasc. Drug. Rev. 20 (I): 27-36.

Blaschke R. J., Rappold G. A.. (2000) SHOX: Growth, Leri-Weill and Turner Syndromes. Trends Endocrinol Metab 11: 227-230.

Bordenave L., Gearges A., Bareille R., Conrad V., Villars F., Arnedee I. (2002) Human hone marrow endothelial cells: a new identified source of B-type natriuretic peptide. Peptides; 23(5): 935-40.

Burger M. R., Burger A. J. (2001) B NP in decompensated heart failure: diagnostic, prognostic and therapeutic potential. CUff. Opin. Inves:tig. Drugs, 2(7): 929-935.

Carel J. C., Mathivon L., GendreI C., Ducret J. P., Chaussain J. L. (1998) Near normalization of final height with adapted doses of growth hormone in Turner's syndrome. J. Clin. Endocrinol. Metab. 83(5): 1462-6.

Cho, Y., Somer, B. G. and Amatya A. (1999) Natriure:tic peptides and their therapeutic potential. Heart Dis. I (5): 305-328.

Chusho H., Ogawa Y., Tamura N., Suda M., Yasoda A., Miyazawa T., Kishimoto I., Komatsu Y., Itoh H., Tanaka K., Saito Y., Garbers D. L., Nakao K. (2000) Genetic models reveal that brain natriuretic peptide can signa]l through different tissue-specific receptor-mediated pathways. Endocrinology, 141(10)″ 3807-13.

Chusho H., Tarnura N., Ogawa Y., Yasoda A., Suda˜.1., Miyazawa T., Nakamura K., Nakao K., Kurihara T., Komatsu Y., Itoh H., Tanaka K., Saito Y., Katsuki M., Nakao K. (2001) Dwarfism and early death in mice lacking C-type natriuretic peptide. Proc Natl Acad Sci USA, 98(7): 4016-21.

Dhingra H., Roongsritong C., Kurtzman N. A. (2002) Brain natriuretic peptide: role in cardiovascular and volume homeostasis. Semin. Nephrol., 22(5): 423-37.

Ellison, J. W., Wardak, Z., Young, M. F., Gehron Robey, P., Laig-Webster, M. and Chiong, W. (1997) PHOG, a candidate gene for involvement in the short stature of Turner syndrome. Hum. Mol. Genet., 6:1341-1347.

Ogawa, Y., Itoh, H., Nakagawa, O., Shirakami, G., Tamura, N., Yoshimasa, T., Nagata, K., Yoshida, N. and Nakao, K. (1995) Characterization of the 5′-flanking region and chromosomal assignment of the human brain natriuretic peptide gene. I. Mol. Med. 73 (9): 457-463.

Rappold G. A., Fukami M., Niesler B., Schiller S., Zurnkeller W., Bettendorf M., Heinrich U., Vlachopapadoupoulou E., Reinehr T., Onigata K., Ogata T. (2002) Deletions of the homeobox gene SHOX (short statu]re homeobox) are an important cause of growth failure in children with short stature. J. Clin. Endocrinol. Metab. 87(3): 1402-1406.

Rao, E., Weiss, B., Fukami, M., Rump, A., Niesler, B., Mertz, A., Muroya, K., Binder, G., Kirsch, S., Winkelmann, M., et al (1997) Pseudoautosomal deletions encompassing a novel homeobox gene cause growth failure in idiopathic short stature and Turner syndrome. Nat. Genet., 16: 54-63.

Rao, E., Blaschke R. J., Marchini, A., Niesler, B., Bwnett, M. and Rappold, G. (2001) The Leri-Weill and Turner Syndrome homeobox ge:ne SHOX encodes a cell type specific transcriptional activator. Human Mol. Gen., 10 (26): 3083-3091.

Rosenfeld R. G., Tesch L. G., Rodriguez-Rigau L. J., McCauley E., Albertsson-Wikland K., Asch R., Cara J., Conte F., Hall J. G., Lippe B., Nagel T. C., Neely E. K., Page D. C., Ranke M., Saenger P., Watkins J. M., Wilson D. M.. (1996) Recommendation for diagnosis, treatment, and management of individuals with Turner syndrome. Endocrinologist 4: 351-358.

Rosenfeld R. G., Attie K. M., Frane J., Brasel J. A., Burstein S., Cara J. F., Chemausek S., Gotlin R. W., Kuntze J., Lippe B. M., Mahoney C. P., Moore w. V., Saenger P. and Johanson A. J. (1998) Growth homlone therapy of Turner's syndrome: beneficial effect on adult height. J. Pediatr. 132(2):319.-24.

Rozen, S. and Skaletsky, H. J. (2000) Primer3 on the ‘WWW for general users and for biologist programmers. In: Krawetz S., Misener S. (eds) Bjojnformatics Methods and Protocols: Methods jn Molecular Bjology. Humana Pr(˜ss, Totowa, N.J., pp 365-386.

Sas T. C. J, de Muinck Keizer-Schrama S. M. P. F., Stlejnen T., Jansen M., Otten B. J., Hoorweg-Nijman J. J. G., Vulsma T., Massa G. G., Rouwe C. W., Reeser H. M., Gerver W. J., Gosen J. J., Rongen-Westerlaken C., Drop S. L. S. (1999) Normalization of height in girls with Turner syndrome after long-term growth hormone treatment: Results of a randomized dose-response trial. J. Clin. Endoctrinol. Metab. 84: 4607-12.

Seilhamer, J. J., Arfsten, A., Miller, J. A., Lundquist, P., Scarborough, R. M., Lewicki, J. A. and Porter, J. G. (1989) Human and canine gene homologs of porcine brain natriuretic peptide. Biochem. Biophys. Res. Commun. 165 (2): 65;0-658 (1989).

Suda M., Ogawa Y., Tanaka K, TamuraN., Yasoda A., Takigawa T., Uehira M., Nishimoto H., Itoh H., Saito Y., Shiota K., Nakao K. (1998) Skeletal overgrowth in transgenic mice that overexpress brain natriuretic peptide. Proc. Natl. Acad. Sci. USA, 95(5): 2337-42.

Weinmann A. S., Farnham P. J. (2002); Identification of unknown targets of human transcription factors using chromatin immunopreacipitation. Methods 26(1): 37-47.

Wilson, D., Sheng, G., Lecuit, T., Dostatni, N. and Desplan, C. (1993) Cooperative dimerization of paired class homeodomains on DNA. (Jenes Dev., 7: 2120-2134. Wilson, D. S., Guenther, B., Desplan, C. and Kuriayan, I. (1995) High resolution crystal structure of a paired (pax) class cooperative homeodomain dimer on DNA. Cell, 82: 709-719.

1. Use of a natriuretic peptide (ANP or BNP) for the preparation of pharmaceutical compositions for the treatment of short stature in a subject being suspected of having a genetic defect in the SHOX gene.

2. Use of a natriuretic peptide (ANP or BNP) in combination with a growth protein for the preparation of pharmaceutical compositions for the treatment of short stature in a subject being suspected of having a genetic defect in the human SHOX gene.

3. Use according to claim 2, wherein said subject is a human subject and said growth hormone is human growth hormone.

4. Use of a natriuretic peptide (ANP or BNP) and a SHOX protein for the preparation of pharmaceutical compositions for the treatment of short stature in a subject being suspected of having a genetic defect in the human SHOX gene.

5. Use according to claim 4 wherein said SHOX protein is a SHOX I protein, especially SHOX A protein or SHOX B protein.

6. Use according to claim 1 for the preparation of pharmaceutical compositions for stimulating or increasing human growth.

7. Use according to claim 1 for the preparation of pharmaceutical compositions for the treatment of patients with idiopathic short stature, patients with Turner syndrome or patients with Leri-Weill syndrome.

8. Use of a natriuretic peptide (ANP or BNP) and a SHOX protein for the preparation of pharmaceutical compositions for the treatment of patients with cardiovascular diseases.

9. Use according to claim 8 wherein the patients are suspected of having a genetic defect in the human growth gene SHOX I.

10. Use according to claim 9 for the preparation of medicaments for the treatment of patients with idiopathic short stature, patients with Turner syndrome or patients with Leri-Weill syndrome.

11. Use according to claim 1 wherein the natriuretic peptide is nesiritide.

12. An article of manufacture comprising packaging material and a pharmaceutical composition comprising a natriuretic peptide (ANP and/or BNP) contained within said packaging material, wherein said pharmaceutical composition is therapeutically effective for treatment of short stature due to a SHOX gene disorder, and wherein said packaging material comprises a label which indicates that said natriuretic peptide can be administered to a subject with a SHOX gene disorder.

13. The article of manufacture of claim 12 further comprising a pharmaceutical composition comprising a growth hormone.

14. The article of manufacture of claim 13 wherein the growth hormone is human growth hormone.

15. The article of manufacture of claim 12 further comprising a pharmaceutical composition comprising a SHOX protein.

16. An article of manufacture comprising packaging material and a pharmaceutical composition comprising a natriuretic peptide (ANP and/or BNP) and a pharmaceutical composition of a SHOX protein contained within said packaging material, wherein said pharmaceutical composition is therapeutically effective for the treatment of cardiovascular diseases, and wherein said packaging material comprises a label which indicates that said natriuretic peptide is effective in treatment of subjects with a SHOX gene disorder.

17. Use of a natriuretic peptide (ANP or BNP) according to claim 1, said patients being identified of having a genetic defect in the human growth gene SHOX using a nucleic acid molecule capable of hybridizing to the SHOX gene.
